(19) United States
(12) Patent Application Publication
(10) Pub. No.: US 2016/0220116 A1
(43) Pub. Date: Aug. 4, 2016

(54) HEALTH ASSESSMENT APPARATUS

(71) Applicant: Gary Waterall, Midlothian (GB)

(72) Inventor: Gary Waterall, Midlothian (GB)

(21) Appl. No.: 14/612,502

(22) Filed: Feb. 3, 2015

(51) Int. Cl. A61B 5/00; A61B 5/107; G09B 23/28; A01K 29/00

(52) U.S. Cl. CPC A61B 5/0053; A01K 29/00; A61B 5/1077; A61B 5/4538; G09B 23/28; A61B 2503/40

(57) ABSTRACT

An assessment apparatus for determining the body condition of an animal, the assessment apparatus comprising a surface, a plurality of protrusions typically in the form or ridges configured on said surface to provide a tactile representation of the ribs of the animal whereby each of the plurality of ridges on said surface is adapted to represent a different possible body condition of the animal. The assessment apparatus is for use when palpating the ribs of an animal to determine the body condition of that animal.

### FIELD OF THE INVENTION

This invention pertains generally to the field of animal health assessment apparatus and in particular apparatus for determining the body condition of an animal by tactile means for use during palpation of the rib cage of the animal. It further relates to a device and method which can be used to determine whether animals are overweight, underweight or at their optimal weight.

### BACKGROUND OF THE INVENTION

It is estimated that around thirty five percent of the total pet population is considered overweight or obese. Obesity in pets may result in a number of health concerns, including osteoarthritis, heart, liver and breathing problems, diabetes, cancer and a compromised immune system.

Pet carers, nutritional organisations and veterinary professionals have indicated that palpating an animal's ribs will provide an indication of whether the animal is overweight, underweight, or at its optimal weight. Many pet owners are aware that their animal's rib cage can be a good indication of the body condition of the animal. When feeling the body of the animal, if they cannot feel the ribs of the animal at all, that is a clear sign that the animal is overweight. Likewise, if the ribs are protruding excessively and clearly visible when looking at the animal, this can be a clear sign that the animal is very underweight. This does however depend on the breed and type of animal.

However, whilst the extremes of body condition are relatively easy to spot, it is difficult to make a proper assessment in most situations. This is where a more thorough physical examination, known as palpation, can help. Palpation is a physical examination whereby a person uses their hands to feel around an area to determine its size, shape, firmness or location. It is in fact, considered by experts to be the most accurate method in determining the body condition of an animal. It is also well-known by those trained in the field that the species and breed of the animal is an important factor to take into consideration when assessing their body condition. The prominence of the ribs and the spacing or grooves between the ribs of the animal's rib cage can be felt during palpation to see if the body condition of the animal is at the optimum condition for such a breed. However the person undertaking the physical examination must have some knowledge as to how the ribs should feel when the animal's body is of the optimum condition. There is a tendency for the layperson to tend toward making an assessment of underweight for a healthy body condition and to determine an animal is good weight when in fact it is overweight.

There are other systems in use for determining the body condition of an animal. These include weighing scales and similar electronic devices; visual dimension determination devices; and further methods which involve the use of specialist equipment such as X-ray machines, callipers, electrodes and lasers. Such devices require some expertise and knowledge on the part of the user.

The prior art shows a number of ways of attempting to solve the problem of determining the body condition of an animal.

U.S. Pat. No. 6,084,941 (Stenstroem) discloses an apparatus which enables visual dimension measurements of entities within a body which are difficult to reach. The device involves the use of an X-rays, an exterior comparison body and a measurement device. The exterior comparison body is such that its shape may match, or closely resemble that of the measurement device. This invention requires that the user have an X-ray of the internal entity, and thus involves a degree of skill and specialist equipment use which is not readily available to a pet owner without consulting a professional.

CA 2268486 (Morissette) describes an invention which employs the use of callipers for longitudinal measurement of the rib cages of livestock animals. This measurement is used as a means of determining the weight of the animal by way of weight graduations and a cursor implemented on one of the two calliper members. Whilst measurement of the ribcage is involved, external individual rib grooves are not considered. WO 2004112605 (Okawa, Umeda) discloses an invention which makes use of electrodes, forming a fixing unit, to be placed in various positions on an animal. The set-up involves measuring the organism impedance by an incorporated circuit (part of a control calculation unit) in order to determine the amount of body fat the animal has. Correct attachment and use of electrodes requires some skill and incorrect attachment may result in erroneous results. This method also requires that electrodes be attached at points on the animal body which are not well covered in fur. This may be challenging in breeds which have long and/or thick fur and thus does not present a universal solution for all animal breeds. It is also an expensive and complex arrangement, that likely to be used by specialists rather than pet or animal owner.

None of the existing methods described herein involve the use of a simple assessment device which aids in rib palpation such that an individual,

untrained in the means of examining an animals' body condition, may easily determine as condition.

### PROBLEM TO BE SOLVED BY THE INVENTION

There is a need for improvements in apparatus and methods that enable individuals to determine whether an animal is overweight, underweight, or at its optimal weight. There is also a need for a device which does not require any technical skill in order to be used and can be used by animal owners. There is a further need for a weight determination device which does not provide any discomfort or cause any anxiety in the animal on which it is being used.

It is an object of the invention to provide a body condition determining device for use with animals.

It is further an object of the invention to provide a body condition determining device which requires no specialist skills or additional training for its use.

It is still a further object of the invention to provide a body condition determining device which can be used for multiple breeds of animal.

### SUMMARY OF THE INVENTION

In accordance with a first aspect of the invention, there is provided an assessment apparatus for determining the body condition of an animal, the apparatus comprising a surface, a plurality of protrusions configured on said surface to provide a tactile representation of one or more characteristics of one or more ribs of an animal whereby the plurality of protrusions on said surface are adapted to represent a range of possible body conditions of the animal.

In a second aspect of the invention, there is provided a method of determining the body condition of an animal, the method comprising the steps of:

feeling the plurality of protrusions of an assessment apparatus as claimed in claim 1 adapted and/or suitable for the particular species and/or breed of animal; feeling by palpation the ribs of the animal; comparing the relative degree of protrusion of one or more protrusions of the apparatus with the degree of protrusion of the ribs of the animal as determined by palpation; selecting from the plurality of protrusions the protrusion or protrusions that most closely represents the extent of protrusion of the ribs of the animal; determining from an indication means provided for the apparatus that indication that indicates the body condition of said animal.

In a third aspect of the invention, there is provided a training apparatus comprising an apparatus as defined above for training people in the determining of the body condition of an animal.

In a fourth aspect of the invention, there is provided a method of training a person in determining the body condition of an animal, the method comprising repeatedly carrying out a method as defined above under instruction.

### ADVANTAGES OF THE INVENTION

The apparatus and method of the present invention provides animal weight status determination means which can be used without prerequisite skills or training. The invention requires the use of palpation of animal ribs in conjunction with use of the device. This is beneficial in that palpation of animal ribs is considered the most accurate means of determining whether an animal is overweight, underweight or at its optimal weight. The invention is applicable for use with multiple species and breeds of animals based on varying prominence of its protrusions. The method employed in conjunction with use of the device requires only a few seconds and thus does not require the animal to be stationary for long periods. Further, the method does not involve preparatory steps for measurement, as has been demonstrated to be commonly required in prior art.

Further embodiments enable users to purchase separate devices with protrusions based on animal species or breeds as required.

### BRIEF DESCRIPTION OF THE DRAWINGS

FIG. 1 illustrates in three-dimensional view a typical arrangement of an assessment apparatus showing a plurality of ridges projected from a surface;

FIG. 2 illustrates the side view of FIG. 1, showing the varying prominence of the ridges;

FIG. 3 illustrates in plan view the assessment apparatus of FIG. 2;

FIG. 4 illustrates the assessment apparatus of FIG. 1, with usage instructions; and

FIG. 5 illustrates a set of assessment apparatuses, for use when palpating different species or breeds of animal.

### DETAILED DESCRIPTION OF THE INVENTION

The assessment apparatus effectively provides a comparison gauge to allow a person to compare the feel of the assessment apparatus with the feel of the ribs or rib cage of an animal, to be able to determine the body condition of the animal. To be able to determine the physical condition of an animal and identify whether the animal is overweight, underweight or of an optimum weight by palpating or feeling the ribs or rib cage of the animal typically relies on the experience and knowledge of that person in making such determination for that particular species and/or breed of animal They need to know the typical feel of the ribs of that particular species and breed of animal when that animal is of an optimum body condition. Without this knowledge and skill it is not currently possible to make an informed decision through palpation alone.

The assessment apparatus of the present invention allows a user to make an assessment of body condition of an animal by providing a tactile representation of the various body conditions of one or more particular species and breeds of animal, to represent by touch how the ribs and the spaces between the ribs would feel if the animal were either overweight, underweight or of an optimum body condition. The person can relate the feel of the animal's ribs and, optionally, the spaces between the ribs to the feel of a plurality of protrusions, preferably ridges, that abut from the surface of the assessment apparatus and determine which of the protrusions on the assessment apparatus, of varying extent, most closely resemble the feel of the ribs of the animal. The measuring gauge may incorporate indication means to indicate what each or certain protrusions represent in terms of body condition of that particular species or breed of animal. Thus by feeling the ribs of the animal, feeling the protrusions of the assessment apparatus and deciding which protrusion or set of protrusions best represents the feel of the rib cage of the animal, the person can determine a value from the indication means that corresponds to that set of protrusions or identify the indication means that states the body condition of that animal.

Preferably, the protrusions vary in prominence or height that they protrude from the surface such that different protrusions are capable of representing the feel of ribs associated with different body condition of an animal.

Preferably, the protrusions are each equally separated from their adjacent protrusions, preferably said separation being determined from a centre point or central line of each protrusion.

Preferably, the protrusions are in the form of ridges or undulations, more preferably ridges and still more preferably linear ridges. Hereinafter, we will refer to ridges, but the more general term, protrusions, may be substituted where the context allows.

Preferably, the plurality of protrusions or ridges are formed on a substantially planar portion of the surface, and more preferably, the surface is planar and still more preferably, the apparatus is a planar element having a plurality of ridges formed thereon.

The protrusions or ridges projecting from the surface of the assessment apparatus may be configured to be in a row and preferably a substantially parallel row of ridges. The ridges are preferably arranged such that each ridge is of different prominence to its adjacent ridge and preferably the prominence of the ridges generally increases from less prominent to more prominent as they are arranged from one end of the apparatus to the other (and vice versa). The less prominent ridges represent the ribs of an animal that is overweight, and the more prominent ridges represent the ribs an animal that is underweight, with the optimum condition of said animal being a certain ridge or certain ridges that are intermediate or somewhere between the most and least prominent ridges. Preferably, at one end of the devices, there is a space with no ridge to represent the situation where a rib is completely submerged by fat and not palpable. The protrusions or ridges need not be arranged in such a row, and may be randomly arranged across the surface. However each of the protrusions or ridges must indicate to a user the body condition that that protrusion or ridge represents.

Preferably, each ridge is of substantially the same curvature, i.e. preferably each ridge defines a curve of substantially similar radius. Preferably, each ridge is separated from its adjacent ridge or ridges by substantially the same distance, preferably the separation being measured not by the distance of planar surface between ridges but the distance between the centre line of each ridge. Consequently, the less prominent ridges may be separated by larger gaps, being areas without protrusions.

The apparatus may be made of any suitable material and is preferably a polymer or polymer-based material, such as a fibre reinforced polymer or filled polymer resin material. Preferably, the apparatus is formed in one piece e.g. by cast or injection moulding.

Optionally, the apparatus may be made of material which is resiliently compressible to the pressure of palpation with the ridges having a more rigid core under the surface whilst the gaps between the ridges have more give.

The assessment apparatus may comprise indication means that forms a scale, or the indication may be a relevant integer, or even words that state the body condition of the animal. The assessment apparatus may also incorporate instructions for use, to ensure that the person using the device does not require any previous knowledge, and can follow the steps indicated to allow them to assess the body condition of an animal.

The assessment apparatus may be specific to one particular animal, such as a pet. The assessment apparatus may incorporate means to allow the person to mark the apparatus, such that repeat measurements can be taken at a later date to monitor changes to the animal's body condition. In an example, a pet dog may be overweight, and showing at a particular point on the scale. The pet dog may be placed on a restricted diet, and re-tested after following the diet for some time. The pet owner can therefore re-measure the pet some weeks later to see if the body condition has improved. The same may be used to monitor an underweight pet, or even to ensure that the pet maintains an optimum body condition.

As mentioned above, in a further aspect, the invention comprises a training device comprising the apparatus as defined above for use in training people in the determination of body condition of an animal. Trainee vets for example can use the device to learn practically how a particular species or breed of animal's ribs should feel when of optimal weight, underweight or overweight. Thus, the device can be used as a training aid.

The invention will now be described in more detail, without limitation, with reference to the accompanying Figures.

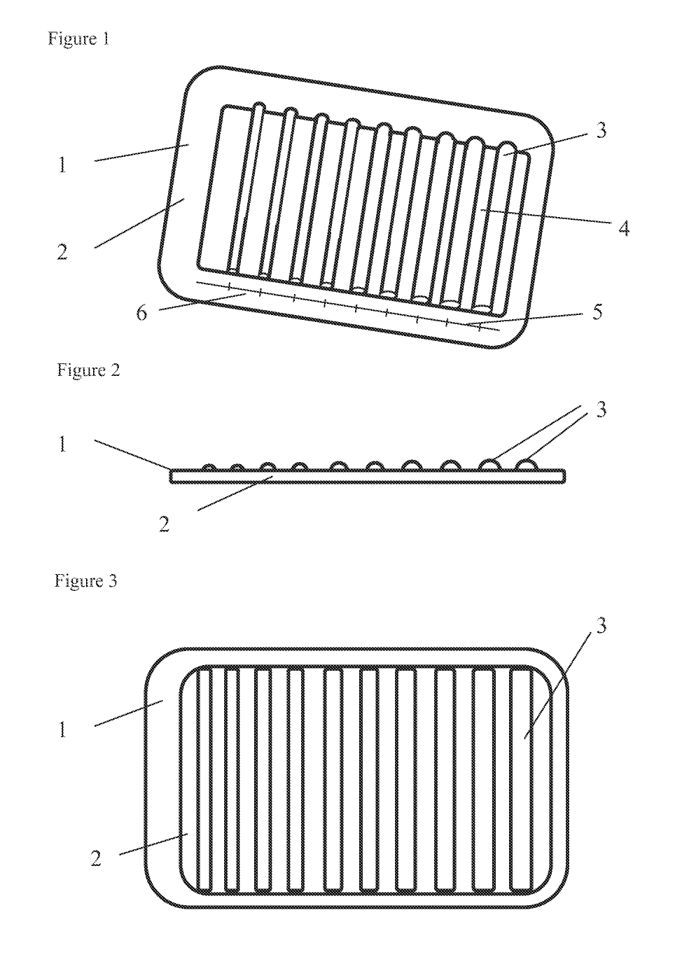

FIG. 1 shows an assessment apparatus 1 comprising a substantially planar surface 2 from which a plurality of ridges 3 project. The plurality of ridges 3 define a plurality of grooves 4 therebetween. The plurality of ridges 3 (and optionally corresponding grooves 4) correspond to different body conditions of an animal, and therefore represent the different feel of the ribs of the animal upon palpation. The veterinary professional, animal carer or animal owner can use the assessment apparatus 1 when palpating the rib cage of the animal to determine whether the animal is of optimum body condition or weight or is overweight or underweight. The ribs of a particular species or breed of animal should feel a certain way if the animal is of optimum condition. If of course the animal is overweight, or carrying excess body fat, the ribs will feel less well defined and the ribs will protrude by a lesser amount, with the space between ribs feeling shallower and perhaps wider. If the animal is underweight, the ribs will be better defined and the space between ribs will feel deeper and, perhaps, narrower. Each of the plurality of ridges 3 that abut from the surface 2 of the assessment apparatus 1 is configured to represent a rib of an animal in a different body condition, whereby the user can use the apparatus to determine the body condition of the animal.

The assessment apparatus 1 of FIG. 1 incorporates a scale 5 with indication means 6, whereby the indication means 6 indicate the body condition of the animal to which each ridge 3 relates. The plurality of ridges 3 are shown in FIG. 1 to increase in prominence from the surface 2 from one side of the surface 2 to the other. The person can therefore move their fingers along the series of ridges 3 in sequence to select the ridge or ridges 3 that best resemble the feel of the ribs of the animal. FIG. 2 shows the assessment apparatus 1 of FIG. 1 in side view, which demonstrate the prominence (or height) of the ridges 3 increasing from one end of the surface 2 to the other. The assessment apparatus 1 may be configured for a particular species and/or breed of animal and the different possible body conditions of that species or breed of animal, or it may be for two or more species or breeds of animal, and the different body conditions of those different species or breeds of animal.

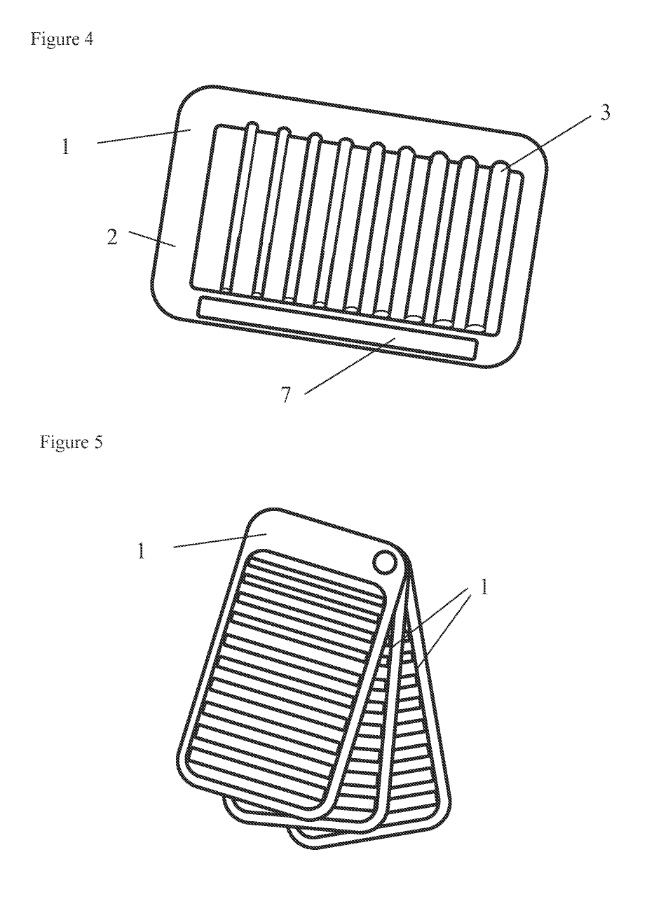

FIG. 3 shows in plan view the assessment apparatus of FIGS. 1 and 2 with the plurality of ridges 3 substantially extending the width of the assessment apparatus 1. The grooves 4 may be formed by the plurality of ridges 3, or may be further defined or shaped to represent more specifically the feel of the rib cage of the animal, not shown. As shown in FIG. 4, the assessment apparatus 1, may also incorporate further indication or instructions 7 as to how to use the apparatus 1 when palpating the rib cage of an animal. The instructions 7 may be on the front surface 2 of the assessment apparatus 1, or may be displayed or printed to the underside of the apparatus 1. The assessment apparatus 1 may also incorporate one or more fixing holes, not shown, for engaging with a hook or nail in a wall, or for affixing to a similar fastening means to mount the measuring gauge 1 to a wall or similar surface. This ensures that the apparatus 1 is on hand whilst being suitable stored when not required.

FIG. 5 shows a plurality of assessment apparatuses 1 shown fastened together at the corner of the surface 2, whereby each measuring gauge may correspond to a different species or breed of animal, or may correspond to a wider range of body conditions of a particular species or breed of animal.

The apparatus 1 may be formed by injection moulding a polymer material, or may comprise any suitable material that allows the ridges 3 to project from a surface 2, and for the feel of said ridges 3 to represent the ribs of an animal. The apparatus may comprise a planar sheet that forms the surface 2, or the surface 2 may be three-dimensional, not shown in the figures, to better represent the curvature of the rib cage of an animal.

The invention has been described with reference to a preferred embodiment. However, it will be appreciated that variations and modifications can be effected by a person of ordinary skill in the art without departing from the scope of the invention.

1. An assessment apparatus for determining the body condition of an animal, the apparatus comprising a surface, a plurality of protrusions configured on said surface to provide a tactile representation of one or more characteristics of one or more ribs of an animal whereby the plurality of protrusions on said surface are adapted to represent a range of possible body conditions of the animal.

2. The apparatus according to claim 1, wherein the protrusions are ridges or undulations formed in or on the surface of the apparatus.

3. The apparatus according to claim 2, wherein the ridges are arranged in a row.

4. The apparatus according to claim 3, wherein the ridges are substantially linear.

5. The apparatus according to claim 1, wherein the each protrusion has an associated curvature and wherein the radius of curvature of each protrusion is substantially the same.

6. The apparatus according to claim 1 wherein each of the protrusions is of substantially equal dimension across.

7. The apparatus according to claim 1, wherein each of the protrusions is equally spaced from each of its adjacent protrusions.

8. The apparatus according to claim 1, wherein the plurality of protrusions are adapted to represent a range of possible body conditions of an animal by having various extents to which different protrusions protrude from the surface.

9. The apparatus according to claim 8, wherein the protrusions are arranged on the surface by degree of extent of protrusion.

10. The apparatus according to claim 1, wherein the surface comprises a substantially planar portion.

11. The apparatus according to claim 1, wherein the surface incorporates an indication means that corresponds to the representative body condition of each of the plurality of protrusions or to one or more of said plurality of protrusions or to groups of said plurality of protrusions.

12. The apparatus according to claim 11, wherein the indication means con rises a scale.

13. The apparatus according to claim 11, wherein the indication means specifies a value of a body condition of an animal.

14. The apparatus according to claim 1, wherein a plurality of protrusions corresponds to characteristics of the ribs of particular species and/or breeds of animal in various body conditions.

15. The apparatus according to claim 1, wherein the plurality of protrusions correspond to characteristics of the ribs of two or more species and/or breeds of animal in various body conditions.

16. The apparatus according to claim 1, which is formed from a polymer material.

17. A method of determining the body condition of an animal, the method comprising the steps of:
feeling the plurality of protrusions of an assessment apparatus, preferably as defined in claim 1, adapted and/or suitable for the particular species and/or breed of animal;
feeling by palpation the ribs of the animal;
comparing the relative degree of protrusion of one or more protrusions of the apparatus with the degree of protrusion of the ribs of the animal as determined by palpation;
selecting from the plurality of protrusions the protrusion or protrusions that most closely represents the extent of protrusion of the ribs of the animal;
determining from an indication means provided for the apparatus that indication that indicates the body condition of said animal.

18. A training apparatus comprising an apparatus as defined in claim 1 for training people in the determining of the body condition of an animal.

19. A method of training a person in determining the body condition of an animal, the method comprising repeatedly carrying out a method as defined in claim 17 under instruction.
